(19) United States
(12) Patent Application Publication
(10) Pub. No.: US 2016/0060589 A1
(43) Pub. Date: Mar. 3, 2016

(54) PARTICLE TRANSPORTING SYSTEM AND METHOD OF OPERATING THE SAME

(71) Applicant: Abnova (Taiwan) Corporation, Taipei (TW)

(72) Inventors: WILBER HUANG, Taipei (TW); Hann-Wen Guan, Taipei (TW)

(21) Appl. No.: 14/840,057

(22) Filed: Aug. 31, 2015

Related U.S. Application Data
(60) Provisional application No. 62/044,335, filed on Sep. 1, 2014.

(51) Int. Cl. C12M 1/26

(52) U.S. Cl. CPC C12M 33/08

(57) ABSTRACT

The present invention provides a particle transporting system including a holder, a vibrator and a tube. The vibrator connects the holder to provide vibration to the holder. The tube spirally surrounds the holder. A method of operating a particle transporting system is provided. The method includes the following steps: (a) providing a particle transporting system as shown above; (b) injecting a sample fluid with plural particles into the tube; and (c) transporting the sample fluid with the particles to a target apparatus with vibration provided by the vibrator.

### CROSS REFERENCE TO RELATED APPLICATIONS

This application claims the benefit of priority of U.S. Provisional Application No. 62/044,335 filed on Sep. 1, 2014, which is incorporated herein by reference.

### BACKGROUND OF THE INVENTION

1. Field of the Invention

The present invention relates to a particle transportation system and a method of operating the same, and more particularly, the present invention is related to a particle transportation system with a vibrator and a spiral tube and a method of operating the same.

2. Description of the Prior Art

If particles in a fluid flow are transported from the top of a funnel-shaped syringe, they tend to aggregate at the bottom of the funnel-shaped syringe due to the force of gravity. Eventually, the particles obstruct the channel where they can't pass through. The situation is even more serious especially in a sticky fluid. For example, cells in blood aggregate more easily at the bottom of the funnel-shaped syringe especially when liquid blood transforms into a semisolid, gel-like state of consistency which is called blood coagulation.

In order to overcome the aforementioned situation, some methods are provided in order to prevent particle aggregation and enhance the movement of particles in a fluid flow. One of the methods is to keep the particles-contained fluids well blended at the bottom of the funnel-shaped syringe by using magnetic stir bars. However, the method is a time consuming and labor intensive process. Moreover, the efficiency for preventing particle aggregation is limited. There is still a need for developing a more convenient method and system with high efficiency to address the aforementioned problems.

### SUMMARY OF THE INVENTION

The present invention therefore provides a particle transporting system so as to avoid above particle aggregation problem.

According to one embodiment, the present invention provides a particle transporting system including a holder, a vibrator and a tube. The vibrator connects the holder to provide vibration to the holder. The tube spirally surrounds the holder. In another embodiment, a method of operating a particle transporting system is provided. The method includes the following steps: (a) providing a particle transporting system as shown above; (b) injecting a sample fluid with plural particles into the tube; and (c) transporting the sample fluid with the particles to a target apparatus with vibration provided by the vibrator.

By both using the vibrator and the spiral tube, the particle transporting system set forth in the present invention can enhance the movement of particles in a fluid flow. Thus, particle aggregation problem in conventional arts can be solved.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 to FIG. 7 show schematic diagrams of the particle transporting system according to different embodiments of the present invention.

FIG. 8 shows a flow chart of the operating method of the particle transporting system according to one embodiment of the present invention.

### DETAILED DESCRIPTION

To provide a better understanding of the presented invention, preferred embodiments will be described in detail. The preferred embodiments of the present invention are illustrated in the accompanying drawings with numbered elements.

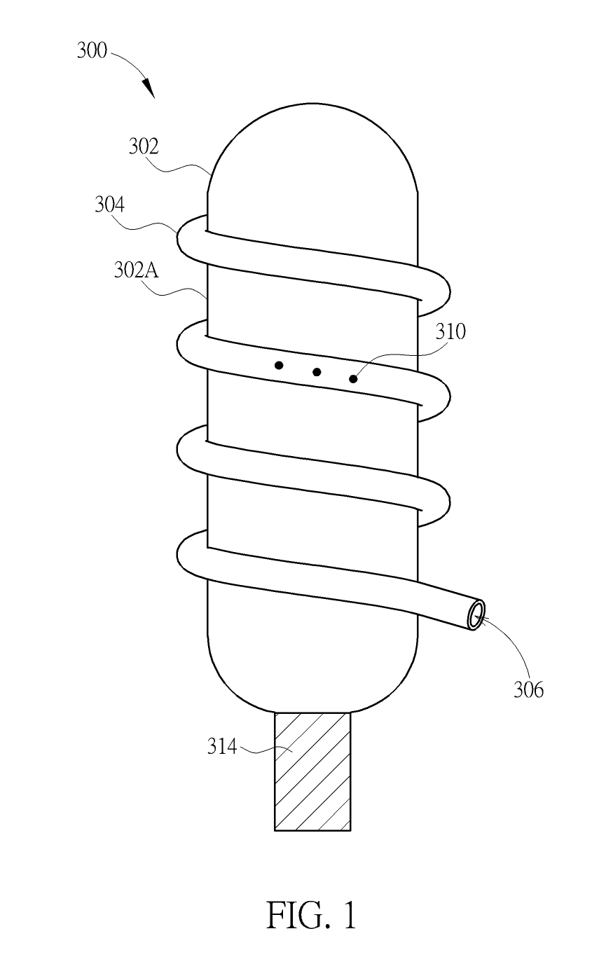

Please refer to FIG. 1, which shows a schematic diagram of the particle transporting system according to one embodiment of the present invention. As shown in FIG. 1, the particle transporting system 300 includes a holder 302, a tube 304, and a vibrator 314. The holder 302 is used to provide the main support for the particle transporting system 300. In the present invention, the holder 302 has an outer surface 302A, such as a cylinder outer surface in one embodiment. The holder 302 can be made of any suitable material such as plastic or metal. The tube 304 spirally surrounds the outer surface 302A of the holder 302 with a gentle slope, clockwise or counterclockwise. In the present invention, the tube 304 has only one opening 306, preferably positioned at lower terminal of the holder 302, to provide the sample fluid in and out. The vibrator 314 is connected to the holder 302 to provide appropriate vibration to the holder 302 as well as the tube 304. The vibrator 314 can be any commercial vibrator that can provide vibration, including but not limited to, a vibrating motor that is improperly balanced. There is an off-centered weight attached to the motor's rotational shaft that causes the motor to wobble. The amount of wobble can be changed by the amount of weight that is attached, the weight's distance from the shaft, and the speed at which the motor spins. In one embodiment, the holder 302 can be hollow in order to accommodate other component, such as the vibrator 314.

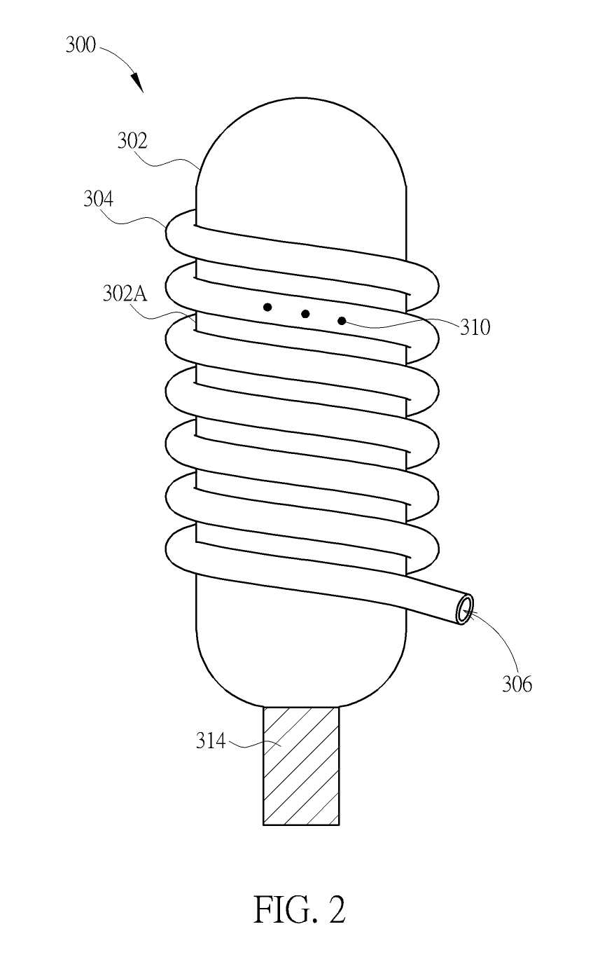

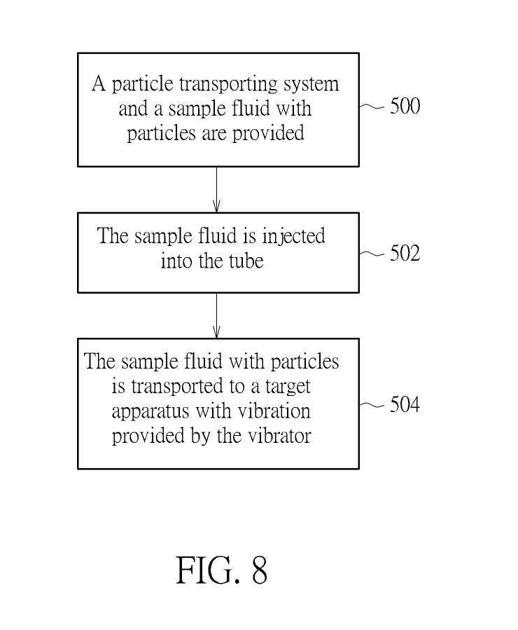

Please see FIG. 8, which shows a flow chart of the using method according to one embodiment of the present invention. Please refer to FIG. 1 and FIG. 8. A particle transporting system and a sample fluid with particles are provided (step 500). The sample fluid can be of any type including solution or vapor and is not limited thereto. In one embodiment, the sample fluid is blood. The particles 310 can be bio-particles or non-bio particles or their combinations. In one embodiment, bio-particles include cells, bacteria or spores, while non-bio particles include beads, magnetic beads, but are not limited thereto. Next, the sample fluid is injected into the tube (step 502). The sample fluid with the particles 310 are delivered, for example, through the opening 306 into the tube 304 where the sample fluid can be accumulated. In one embodiment, the volume of the tube 304 can be adjusted so as to accumulate more sample fluid. For example, the inner diameter of the tube 304 can be altered. Alternatively, as shown in FIG. 2, the spiral tube 304 can surround the holder 302 with more laps so as to gain a greater volume.

The sample fluid with particles 310 is transported to a target apparatus with vibration provided by the vibrator (step 504). The target apparatus can be any apparatus used to collect or analysis the particles 310, such as cell flow analyzer, fluorescence spectrometry, and is not limited thereto. Since the spiral tube 304 has a gentle slope and thus decreases the moving rate of the particles 310 contained therein due to the force of gravity. In addition, by using the vibrator 314, the friction between the particles 310 in a fluid flow and the inner surfaces of the spiral tube 304 can be reduced. Thus, the particles 310 in a fluid flow can move without aggregation. It is understood the slope of the spiral tube 304 can be adjusted depending on the size of the particles, the weight of the particles, the parameters of the fluid . . . , or other factors. As shown in FIG. 1 and FIG. 2, a gentle slope is provided in the embodiment of FIG. 2 by making the tube 304 surrounding more laps. It is also possible to change the shape of the outer surface 302A of the holder 302 to change the slope.

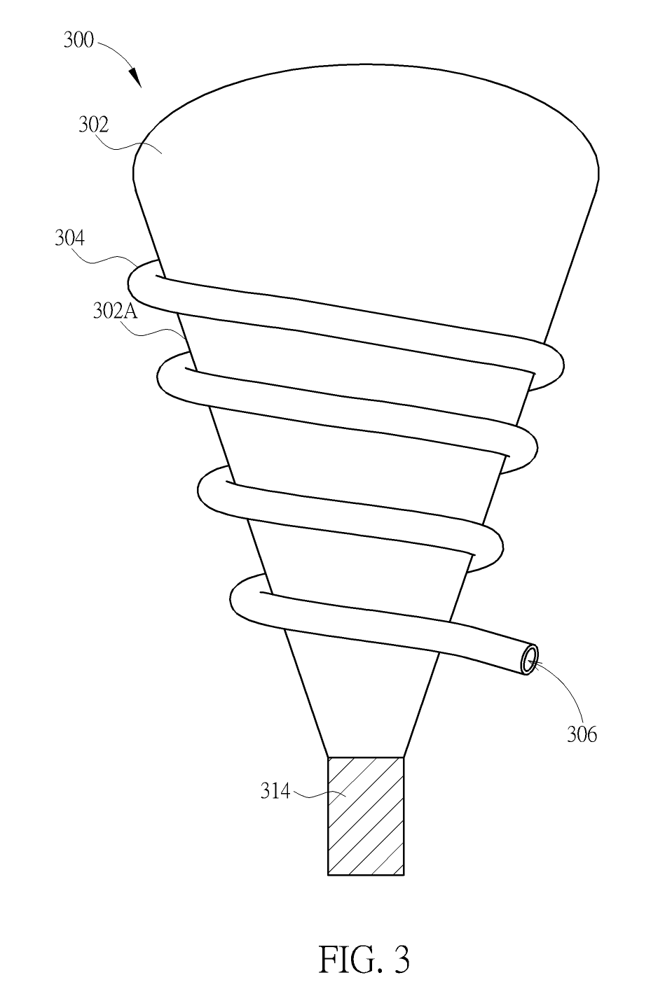

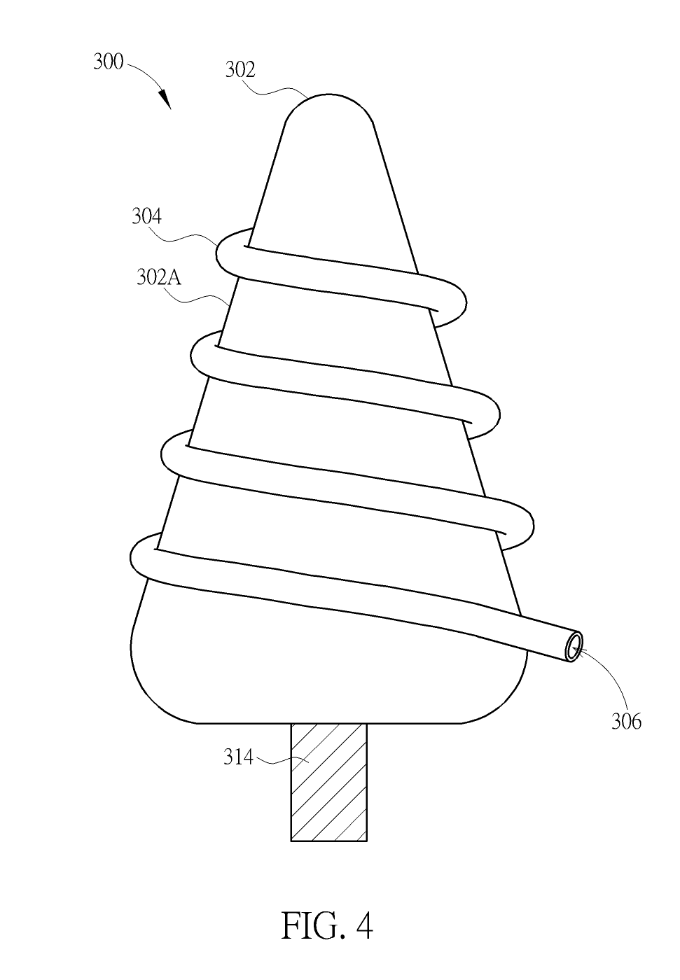

Please see FIG. 3 and FIG. 4, which shows schematic diagrams of the particle transporting system according to two embodiments of the present invention. As shown in FIG. 3, the outer surface 302A of the holder 302 has a syringe shape in which the crossing section thereof shrinks from top to bottom. On the contrary, as shown in FIG. 4, the crossing section of the holder 302 shrinks from bottom to top.

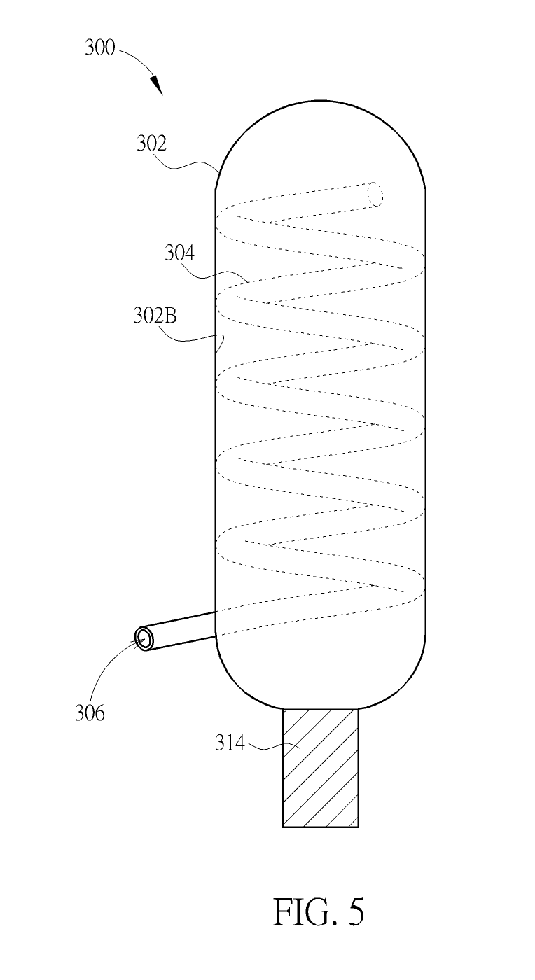

Please see FIG. 5, which shows a schematic diagram of the particle transporting system according to one embodiment of the present invention. As shown in FIG. 5, the holder 302 is hollow and has an inner surface 302B wherein the tube 304 spirally surrounds the inner surface 302B. In this embodiment, the hollow holder 302 is much easy to vibrate because it has less weight. Similarly, the shape of the inner surface 302B can be altered so as to adjust the slope of the tube 304.

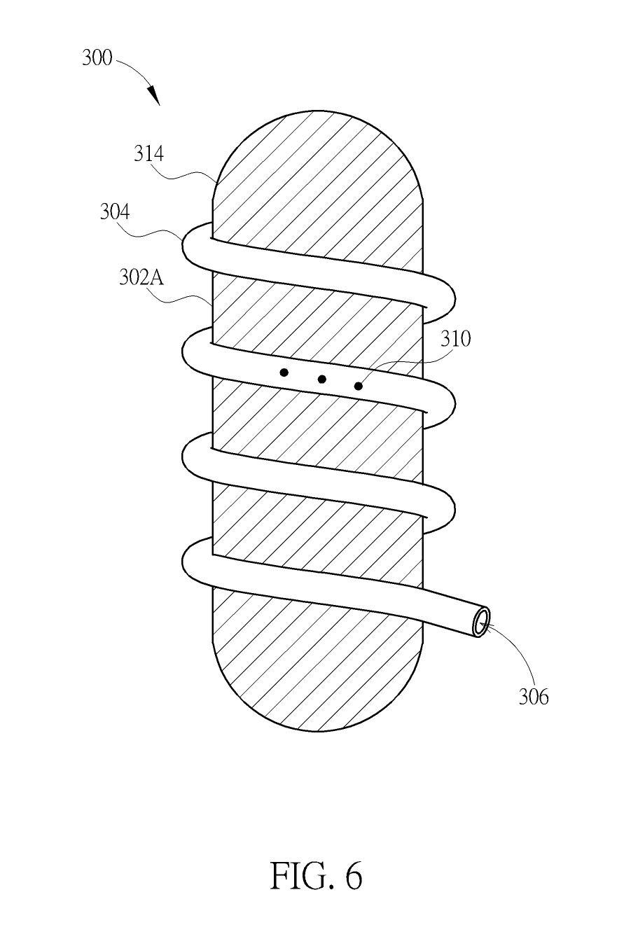

Please see FIG. 6, which shows a schematic diagram of the particle transporting system according to one embodiment of the present invention. As shown in FIG. 6, the vibrator 314 and the holder 302 are monolithic. That is, the vibrator 314 or a part of the vibrator 314 can itself serves as the holder and the tube 304 directly surrounds the vibrator 314 or a part of the vibrator 314. It is understood that this embodiment can also incorporated into any previous embodiments.

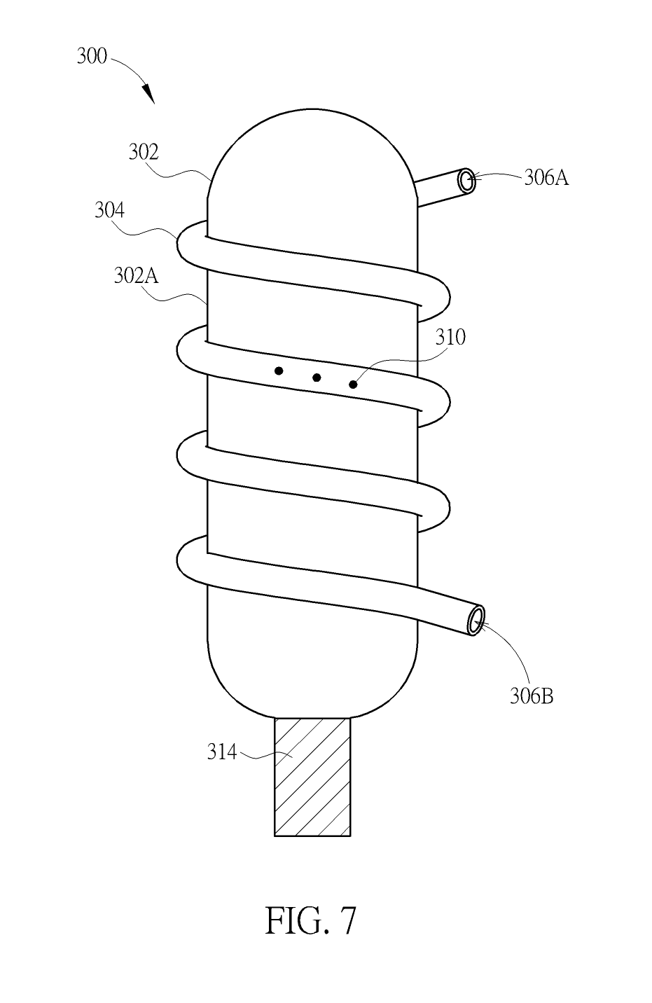

Please see FIG. 7, which shows a schematic diagram of the particle transporting system according to one embodiment of the present invention. As shown in FIG. 7, the spiral tube 304 has two openings 306A and 306B. In this embodiment, the opening 306A can connect to a sample fluid with particles 310 and the opening 306B is connected to a target apparatus. When the sample fluid flows through the opening 306A into the tube 304, the vibrator 314 starts provide vibration, and then the particles 310 can flow to the target apparatus without aggregation. Similarly, this embodiment can also be incorporated into any previous embodiments.

### Example 1

### Materials and Methods

The holder is a cylinder which is made of polystyrene and has an outer diameter of 10 mm.

The vibrator has the following characteristics:

### Operating Voltage: 2.5˜3.5V DC

### Starting Voltage: 1.5 V DC Max

### Rated Speed: 10000±2000 rpm

### Rated Current: 70 mA Max

### Vibration Displacement: 1.5 mm

### Vibration Frequency: 10 to 55 Hz

### Outer Diameter (O.D.): 6 mm

The spiral tube fully surrounds an outer surface of the holder and is a cylindrical pipe which has the following characteristics: Material: Polytetrafluoroethylene (PTFE)

Internal Diameter (I.D.): 0.04 inch
Outer diameter (O.D.): 0.0625 inch

### Length: 170 mm

### Experiment

Fluorescent PC9 cells (about 300 cells) with PBS (100 μl) or with PBMC-contained Wash Medium (100 μl, RPMI+5% FBS; peripheral blood mononuclear cells are prepared from 2 ml blood using Leucosep method) are delivered by the aforementioned spiral tube which surrounds the aforementioned vibrator. Flush the cells with 0.3 ml Wash Medium (RPMI+5% FBS) at flow of 1.2 ml/hr and then wash with PBS at flow of 4.8 ml/hr for 12 minutes. The fluorescent PC9 cells are eventually captured by antibodies coated on chip (Capture Antibody: biotinylated mouse anti-EpCAM (250 μg/ml) coating for 1 hr at room temperature (25° C.), and then wash three times with 100 μl PBS).

The fluorescent PC9 cells captured by the aforementioned antibodies and the uncaptured fluorescent PC9 cells in the waste tank are both counted to evaluate the recovery rate. Recovery rate=The fluorescent PC9 cells captured by antibodies and those uncaptured in waste tank/total fluorescent PC9 cells initially added.

### Results

The recovery rate of the fluorescent PC9 cells with PBS is 93.8% (305/325). The recovery rate of the fluorescent PC9 cells with PBMC is 91.7% (321/350). The results show that the system comprising a vibrator and a spiral tube and the method described above can enhance the movement of particles in a fluid flow and thus the recovery rate is high.

### Control Experiment

### Materials and Methods

Materials and methods are the same as Example 1 except that a 1 ml funnel-shaped syringe and a magnetic stir bar are substituted for the holder, the vibrator and the spiral tube.

### Results

The recovery rate of the fluorescent PC9 cells with PBS is 73.3% (257/345). The recovery rate of the fluorescent PC9 cells with PBMC is 68.1% (203/298). The results show that the recovery rate is low in comparison with that of Example 1.

It is proved that the particle transporting system can enhance the movement of cells in a fluid flow without aggregation.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A particle transporting system, comprising:
a holder;
a vibrator connecting the holder to provide vibration to the holder; and
a tube spirally surrounding the holder.

2. The particle transporting system according to claim 1, wherein the holder and the vibrator are monolithic.

3. The particle transporting system according to claim 1, wherein the tube spirally surrounds an outer surface of the holder.

4. The particle transporting system according to claim 1, wherein the tube spirally surrounds an inner surface of the holder.

5. The particle transporting system according to claim 1, wherein the tube has only one opening.

6. The particle transporting system according to claim 1, wherein the tube has two openings at its two terminals.

7. The particle transporting system according to claim 1, wherein the holder has a substantially cylinder shape.

8. The particle transporting system according to claim 1, wherein the holder has a cross section shrinking from top to bottom.

9. The particle transporting system according to claim 1, wherein the holder has a cross section shrinking from bottom to top.

10. The particle transporting system according to claim 1, wherein the tube can accumulate or deliver a sample fluid with a plurality of particles.

11. The particle transporting system according to claim 10, wherein the particles comprises cells, bacteria or spores.

12. The particle transporting system according to claim 10, wherein the particles comprises beads.

13. The particle transporting system according to claim 10, wherein the sample fluid is blood.

14. A method of operating a particle transporting system, comprising the following steps:
(a) providing a particle transporting system as in claim 1;
(b) injecting a sample fluid with plural particles into the tube; and
(c) transporting the sample fluid with the particles to a target apparatus with vibration provided by the vibrator.

15. The method of operating a particle transporting system according to claim 14, wherein the tube has only one opening and in step (b) the sample fluid is injected through the opening, and in step (c) the sample fluid is transported to the target apparatus through the said opening.

16. The method of operating a particle transporting system according to claim 14, wherein the tube has a first opening and a second opening, and in step (b) the sample fluid is injected through the first opening, and in step (c) the sample fluid is transported to the target apparatus through the second opening.

17. The method of operating a particle transporting system according to claim 14, wherein the holder and the vibrator are monolithic.

18. The method of operating a particle transporting system according to claim 14, wherein the particles comprises cells, bacteria or spores.

19. The method of operating a particle transporting system according to claim 14, wherein the particles comprises beads.

20. The method of operating a particle transporting system according to claim 14, the sample fluid is blood.
